(19) United States
(12) Patent Application Publication
(10) Pub. No.: US 2025/0339098 A1
(43) Pub. Date: Nov. 6, 2025

(54) BLOOD PRESSURE MEASUREMENT DEVICE

(71) Applicant: OMRON HEALTHCARE Co., Ltd., Kyoto (JP)

(72) Inventors: Masaki HARADA, Kyoto (JP); Yoshihiko SANO, Kyoto (JP); Yoshihide TOKKO, Kyoto (JP); Kazuya MURASE, Kyoto (JP); Sayuri KONOBE, Kyoto (JP); Shingo ISHIBASHI, Kyoto (JP)

(21) Appl. No.: 19/271,251

(22) Filed: Jul. 16, 2025

Related U.S. Application Data
(63) Continuation of application No. PCT/JP2023/038727, filed on Oct. 26, 2023.

(30) Foreign Application Priority Data
Feb. 1, 2023 (JP) 2023-014291

(51) Int. Cl. A61B 5/00; A61B 5/0205; A61B 5/022

(52) U.S. Cl. CPC A61B 5/681; A61B 5/0205; A61B 5/02233; A61B 5/6831; A61B 2560/0462; A61B 2562/0247; A61B 2562/06

(57) ABSTRACT

A blood pressure measurement device to be worn on a wrist by a band includes: a device body including a case, a pump provided in the case, a pressure sensor, a fluid circuit to be connected to the pump and the pressure sensor, and a sensor unit provided on a rear surface of the case facing toward the wrist; a first pressing cuff connected to the fluid circuit and fixed to one end of the rear surface of the case in one direction away from the sensor unit; and a second pressing cuff connected to the fluid circuit, fixed to the other end of the rear surface of the case in the one direction away from the sensor unit, and fluidly connected to the first pressing cuff. The first pressing cuff and the second pressing cuff are wound around the wrist by the band.

### CROSS-REFERENCE TO RELATED APPLICATIONS

This application is the U.S. national stage application filed pursuant to 35 U.S.C. 365(c) and 120 as a continuation of International Patent Application No. PCT/JP2023/038727, filed Oct. 26, 2023, which application claims priority to Japanese Patent Application No. 2023-014291, filed Feb. 1, 2023, which applications are incorporated herein by reference in their entireties.

### TECHNICAL FIELD

The present invention relates to a blood pressure measurement device.

### BACKGROUND ART

In recent years, blood pressure measurement devices used for measuring blood pressure are being utilized as a means for checking a health state not only at a medical facility but also at home. A blood pressure measurement device detects vibration of the artery wall to measure blood pressure by, for example, inflating and contracting a cuff wound around the wrist or the like of a living body and detecting the pressure in the cuff by using a pressure sensor.

Further, as disclosed in WO 2012/018029 and JP 2020-103628 A, a wearable blood pressure monitor to be worn on a wrist is known as a blood pressure measurement device. In such a blood pressure measurement device, a cuff is disposed on the rear surface of the device body in order to secure a compression area of the wrist. For this reason, various sensors such as an electrocardiogram (ECG) sensor and a photoplethysmography (PPG) sensor cannot be disposed on the rear surface of the device body of the blood pressure measurement device.

Further, as disclosed in JP 62-292137 A, a known blood pressure measurement device has a configuration in which an air chamber is provided on a back side of a device body, and a cuff joined to a belt is connected to both sides of the device body through this air chamber.

### CITATION LIST

### Patent Literature

Patent Literature 1: WO 2012/018029; Patent Literature 2: JP 2020-103628 A; Patent Literature 3: JP 62-292137 A

### SUMMARY OF INVENTION

### Technical Problem

In the blood pressure measurement devices described in WO 2012/018029 and JP 2020-103628 A, in order to provide various sensors on the rear surface of the device body, it is necessary to eliminate the cuff from the rear surface of the device body. However, if the cuff is not provided on the rear surface of the device body, the compression area becomes small, and thus the measurement accuracy of the blood pressure measurement device decreases.

In the blood pressure measurement device described in JP 62-292137 A, since the air chamber is provided on the rear surface of the device body, it is difficult to dispose a sensor unit on the rear surface of the device body.

Therefore, an object of the present invention is to provide a blood pressure measurement device having satisfactory measurement accuracy even when a sensor unit is disposed on a rear surface of a device body.

### Solution to Problem

According to an aspect, a blood pressure measurement device to be worn on a wrist by a band is provided, the blood pressure measurement device including: a device body including a case, a pump provided in the case, a pressure sensor, a fluid circuit to be connected to the pump and the pressure sensor, and a sensor unit provided on a rear surface of the case facing toward the wrist; a first pressing cuff connected to the fluid circuit and fixed to one end of the rear surface of the case in one direction away from the sensor unit; and a second pressing cuff connected to the fluid circuit, fixed to the other end of the rear surface of the case in the one direction away from the sensor unit, and fluidly connected to the first pressing cuff, in which the first pressing cuff and the second pressing cuff are wound around the wrist by the band, and when the blood pressure measurement device is worn on a wrist having an expected maximum circumference, at least one of the first pressing cuff and the second pressing cuff faces at least one of a radial artery and an ulnar artery passing through the wrist, and the first pressing cuff and the second pressing cuff overlap.

According to this aspect, since the rear surface of the case is provided with the sensor unit, the first pressing cuff, and the second pressing cuff, the sensor unit can acquire information, and the first pressing cuff and second pressing cuff that are inflated can compress the wrist. Further, in the blood pressure measurement device, the sensor unit is provided in the case, so that even if the first pressing cuff and the second pressing cuff cannot press the wrist in the case, the first pressing cuff and the second pressing cuff can overlap, and thus it is possible to suitably compress the wrist.

Therefore, even if provided with the sensor unit on the rear surface of the device body, the blood pressure measurement device has satisfactory measurement accuracy.

The blood pressure measurement device of the aspect described above includes a sensing cuff disposed on a side of one of the first pressing cuff and the second pressing cuff, the side facing toward the wrist, the sensing cuff being connected to the fluid circuit.

According to this aspect, the blood pressure measurement device can measure the blood pressure by the sensor disposed on the side of one of the first pressing cuff and the second pressing cuff, the side facing toward the wrist.

In the blood pressure measurement device of the aspect described above, the sensor unit includes at least one of a biometric sensor and a charging terminal.

According to this aspect, in the blood pressure measurement device, since the biometric sensor or the charging terminal is provided on the rear surface of the device body, the biometric sensor or the charging terminal is not provided at a position far from the device body, and the degree of freedom in arrangement is increased. Further, since the sensor unit is provided in the case of the device body having high rigidity, and can come into contact with the wrist when the blood pressure measurement device is worn on the wrist, the biological information can be suitably acquired by the biometric sensor. Further, the charging terminal is provided in the sensor unit, so that the charging terminal is exposed to the outside when the blood pressure measurement device is removed from the wrist, and thus charging using the charging terminal is facilitated.

In the blood pressure measurement device of the aspect described above, the first pressing cuff and the second pressing cuff are fluidly connected to each other through the fluid circuit provided in the device body.

According to this aspect, in the blood pressure measurement device, since the first pressing cuff and the second pressing cuff can be fluidly connected to each other through the fluid circuit provided in the device body, the arrangement of the first pressing cuff and the second pressing cuff on the rear surface can be simplified.

In the blood pressure measurement device of the aspect described above, the first pressing cuff and the second pressing cuff are fluidly connected to each other on the rear surface, while avoiding the sensor unit.

According to this aspect, in the blood pressure measurement device, since the first pressing cuff and the second pressing cuff are connected to each other, while avoiding the sensor unit, it is possible to prevent the first pressing cuff and the second pressing cuff from interfering with the sensor unit and hindering the function of the sensor unit.

The blood pressure measurement device of the aspect described above includes a cuff cover fixed to the rear surface, having an opening formed for disposing the sensor unit, and covering a part of the first pressing cuff and a part of the second pressing cuff.

According to this aspect, in the blood pressure measurement device, the cuff cover covers the rear surface except for the sensor unit, whereby the cuff cover covers a part of the first pressing cuff and a part of the second pressing cuff. Therefore, in the first pressing cuff and the second pressing cuff, a portion positioned on the rear surface is protected by the cuff cover, and movement in a direction away from the rear surface is restricted.

In the blood pressure measurement device of the aspect described above, the device body includes a vent formed at a position on the rear surface away from the sensor unit.

According to this aspect, in the blood pressure measurement device, ventilation between the inside and the outside of the device body is possible, and moisture can be prevented from entering the inside. Further, since the vent is provided at a position away from the sensor unit, the vent is covered and protected by the cuff cover.

In the blood pressure measurement device of the aspect described above, the sensor unit includes at least one of a PPG sensor, an SpO2 sensor, and an electrocardiographic electrode.

According to this aspect, the blood pressure measurement device can acquire, as biological information, at least one of the heart rate, the saturation, and the electrocardiogram waveform in addition to the blood pressure.

### Advantageous Effects Of Invention

According to the present invention, it is possible to provide a blood pressure measurement device having satisfactory measurement accuracy even when a sensor unit is disposed on a rear surface of a device body.

### BRIEF DESCRIPTION OF DRAWINGS

FIG. 1 is a perspective view illustrating a configuration of a blood pressure measurement device according to an embodiment of the present invention;

FIG. 2 is a side view illustrating the configuration of the blood pressure measurement device;

FIG. 3 is a side view illustrating the configuration of the blood pressure measurement device in a state of being worn on a wrist;

FIG. 4 is a block diagram illustrating the configuration of the blood pressure measurement device;

FIG. 5 is a cross-sectional view illustrating a configuration of a main portion of the blood pressure measurement device;

FIG. 6 is a plan view illustrating the configuration of the main portion of the blood pressure measurement device from a rear surface side of a device body;

FIG. 7 is a plan view illustrating the configuration of the main portion of the blood pressure measurement device with some configurations omitted, when viewed from the rear surface side of the device body;

FIG. 8 is an exploded perspective view illustrating a configuration of the device body;

FIG. 9 is an exploded perspective view illustrating the configuration of the device body;

FIG. 10 is a block diagram illustrating an example of a fluid circuit of the blood pressure measurement device;

FIG. 11 is a plan view illustrating the configuration of the main portion of the blood pressure measurement device according to another embodiment of the present invention, when viewed from a rear surface side of a device body;

FIG. 12 is a side view illustrating the configuration of the blood pressure measurement device according to another embodiment of the present invention;

FIG. 13 is a plan view illustrating the configuration of the main portion of the blood pressure measurement device according to another embodiment of the present invention, when viewed from a rear surface side of a device body;

FIG. 14 is a block diagram illustrating an example of the fluid circuit of the blood pressure measurement device; and,

FIG. 15 is a block diagram illustrating an example of the fluid circuit of the blood pressure measurement device.

### DESCRIPTION OF EMBODIMENTS

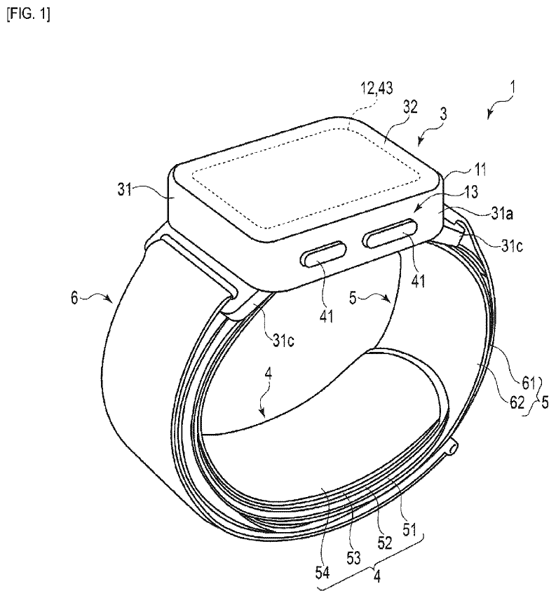

Hereinafter, an example of a blood pressure measurement device 1 according to an embodiment of the present invention will be described with reference to FIGS. 1 to 10.

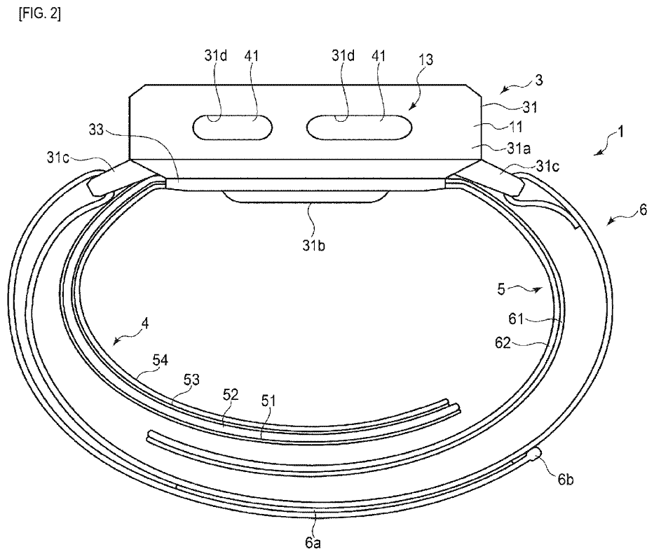

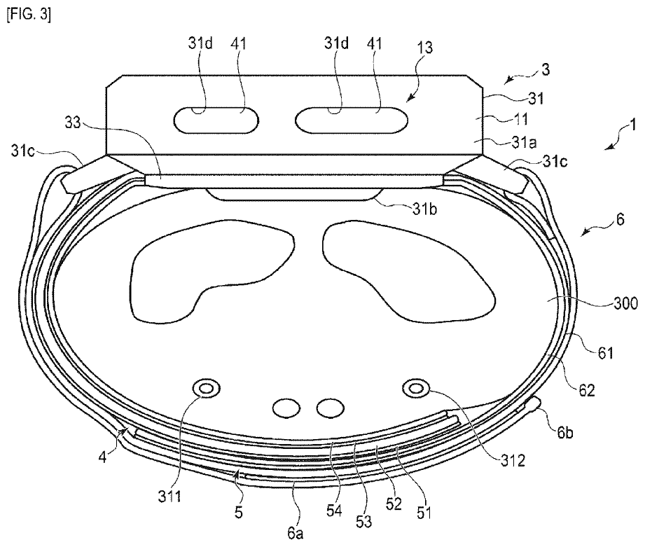

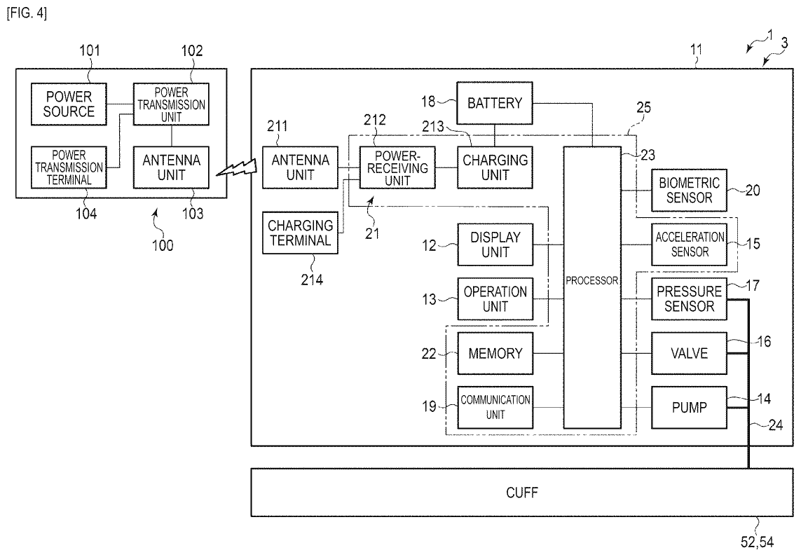

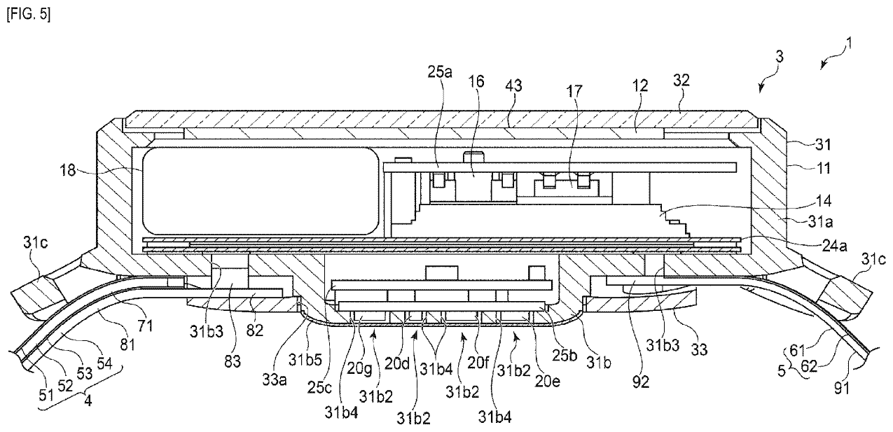

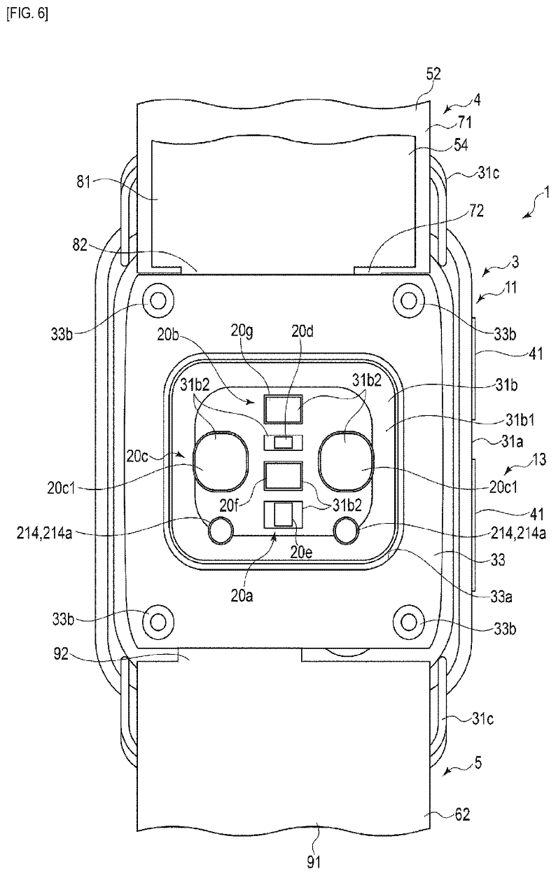

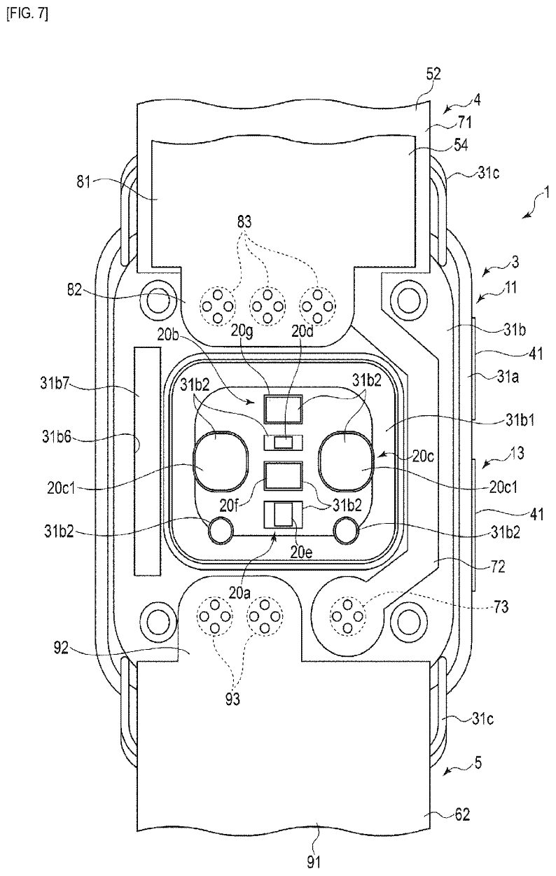

FIG. 1 is a perspective view illustrating a configuration of the blood pressure measurement device 1 according to the embodiment of the present invention. FIG. 2 is a side view illustrating the configuration of the blood pressure measurement device 1. FIG. 3 is a side view illustrating the configuration of the blood pressure measurement device 1 in a state of being worn on a wrist 300. FIG. 4 is a block diagram illustrating the configuration of the blood pressure measurement device 1. FIG. 5 is a cross-sectional view illustrating a configuration of a device body 3, a portion of a first cuff structure 4, and a portion of a second cuff structure 5 of the blood pressure measurement device 1. FIG. 6 is a plan view illustrating the configuration of the device body 3, a portion of the first cuff structure 4, and a portion of the second cuff structure 5 of the blood pressure measurement device 1. FIG. 7 is a plan view illustrating the configuration of the device body 3, a portion of the first cuff structure 4, and a portion of the second cuff structure 5 of the blood pressure measurement device 1 with a cuff cover 33 omitted.

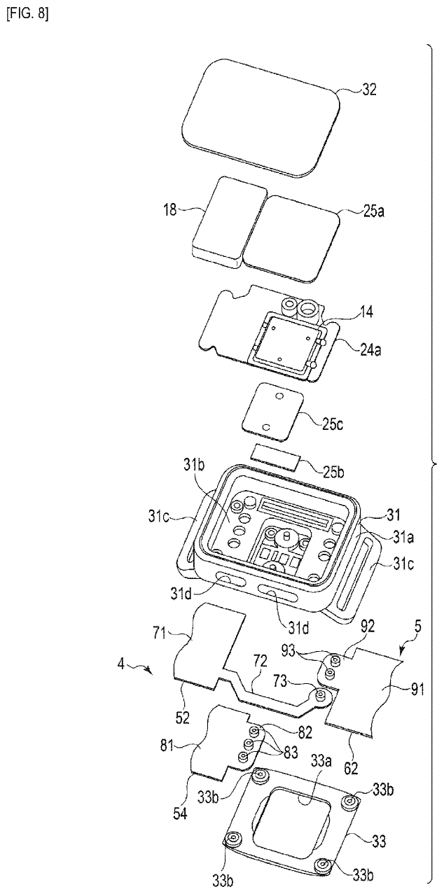

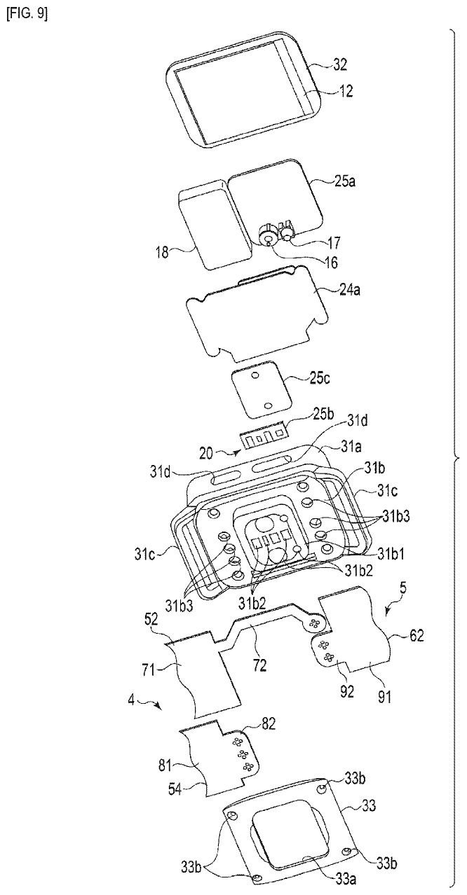

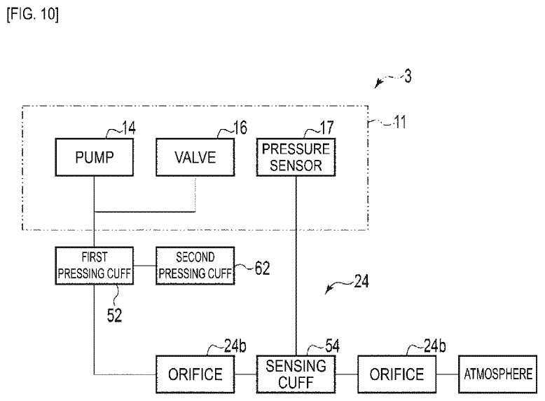

FIG. 8 is an exploded perspective view illustrating the configuration of the device body 3, when viewed from an upper surface side. FIG. 9 is an exploded perspective view illustrating the configuration of the device body 3, when viewed from a lower surface side. FIG. 10 is a block diagram illustrating an example of a fluid circuit 24 of the blood pressure measurement device 1.

As illustrated in FIGS. 1 to 3, the blood pressure measurement device 1 includes, for example, the device body 3, the first cuff structure 4, the second cuff structure 5, and a band 6. The blood pressure measurement device 1 is configured such that the first cuff structure 4 and the second cuff structure 5 extend from the device body 3 in opposite directions of the device body 3, with the band 6 covering the first cuff structure 4 and the second cuff structure 5, and is formed fixable to the wrist 300 that is a living body by the band 6.

As illustrated in FIGS. 1 to 4, the device body 3 includes, for example, a case 11, a display unit 12, an operation unit 13, a pump 14, an acceleration sensor 15, a valve 16, a pressure sensor 17, a battery 18, a communication unit 19, biometric sensors 20, a charging circuit unit 21, a memory 22, a processor 23, the fluid circuit 24, and a substrate 25.

The case 11 is a case that accommodates components of the device body 3. The case 11 accommodates, for example, the display unit 12, the operation unit 13, the pump 14, the acceleration sensor 15, the valve 16, the pressure sensor 17, the battery 18, the communication unit 19, the biometric sensors 20, the charging circuit unit 21, the memory 22, the processor 23, the fluid circuit 24, and the substrate 25.

The case 11 includes, for example, an outer case 31, a windshield 32 covering an upper opening of the outer case 31, and a cuff cover 33 provided below the outer case 31.

The outer case 31 is formed in a bottomed tubular shape such as, for example, a cylindrical, rectangular tubular, or polygonal tubular shape with a bottom portion. In the present embodiment, an example is illustrated in which the outer case 31 is formed in a bottomed rectangular tubular shape. As a specific example, the outer case 31 includes a peripheral wall portion 31a having a rectangular tubular shape, a bottom portion 31b provided at the peripheral wall portion 31a, and a pair of loop portions 31c provided at a pair of surfaces among four outer peripheral surfaces of the peripheral wall portion 31a.

In the peripheral wall portion 31a, for example, an opening 31d in which part of the operation unit 13 is disposed is formed. For example, the opening 31d is formed in one surface of the peripheral wall portion 31a that is different from the two surfaces provided with the pair of loop portions 31c. Further, for example, two of the openings 31d are formed in one surface of the peripheral wall portion 31a.

The bottom portion 31b constitutes a rear surface (bottom portion 31b) of the case 11 (outer case 31) facing the wrist 300. The bottom portion 31b, for example, partially projects facilitating coming into contact with the wrist 300. For example, the bottom portion 31b includes a projection portion 31b1 formed by a center side of an outer surface side being projected in a rectangular shape and then a center side of an inner surface side of the bottom portion 31b being recessed in a rectangular shape. Further, in the bottom portion 31b, a plurality of window portions 31b2 are formed in which the biometric sensors 20 are disposed, and a plurality of hole portions 31b3 are formed in which connection portions 73 and 83 described below are disposed as members fluidly connecting the first cuff structure 4 and the second cuff structure 5 to the pump 14.

The biometric sensors 20 are disposed on the inner surface side of the projection portion 31b1. The projection portion 31b1 constitutes a sensor unit including at least one of the biometric sensors 20 and a charging terminal 214 disposed on the rear surface of the case 11. Here, the biometric sensors 20 and the charging terminal 214 being disposed on the rear surface of the case 11 mean that some of the components constituting the biometric sensors 20 are exposed on the rear surface of the case 11, or are disposed on the rear surface of the case 11 with a member (for example, a cover 31b5 described below) other than the first cuff structure 4 and the second cuff structure 5 interposed therebetween.

The window portions 31b2 are formed in the projection portion 31b1. For example, as illustrated in FIG. 5, the window portions 31b2 are formed by a plurality of openings 31b4 formed at a plurality of locations of the projection portion 31b1 and by the cover 31b5 having transparency, made of glass, a resin material, or the like, and covering these openings 31b4. The cover 31b5 covers, for example, an outer surface of the projection portion 31b1.

The plurality of hole portions 31b3 are respectively formed between the projection portion 31b1 and each of the loop portions 31c in the bottom portion 31b. In the present embodiment, three hole portions 31b3 are formed between the projection portion 31b1 and one loop portion 31c, and three hole portions 31b3 are formed between the projection portion 31b1 and the other loop portion 31c. These three hole portions 31b3 are arranged in one direction, for example, a direction orthogonal to the opposing direction of the pair of loop portions 31c. That is, in the bottom portion 31b, which is the rear surface of the case 11, the three hole portions 31b3 in which the partial configuration of the first cuff structure 4 is fixed to one end portion in one direction are arranged at positions away from the projection portion 31b1, and the three hole portions 31b3 in which the other partial configuration of the first cuff structure 4 and the second cuff structure 5 are fixed to the other end portion in the one direction are arranged away from the projection portion 31b1 on another side in the one direction.

Further, a vent 31b6 having a slit shape elongated in the one direction is formed in the bottom portion 31b, and the bottom portion 31b includes a waterproof moisture-permeable sheet 31b7 covering this vent 31b6. The waterproof moisture-permeable sheet 31b7 is formed so as to prevent moisture from entering the case 11 from the outside through the vent 31b6 of the bottom portion 31b and to facilitate ventilation between the inside and the outside of the case 11. Here, the vent 31b6 and the waterproof moisture-permeable sheet 31b7 are provided at positions not overlapping the first cuff structure 4 and the second cuff structure 5 fixed to the bottom portion 31b, that is, at positions away from the first cuff structure 4 and the second cuff structure 5. For example, in the direction orthogonal to the opposing direction of the pair of loop portions 31c, a flow path body 72 of a first pressing cuff 52, to be described below, of the first cuff structure 4 is provided adjacent to one side of the projection portion 31b1, and the vent 31b6 and the waterproof moisture-permeable sheet 31b7 are provided adjacent to the other side of the projection portion 31b1.

The loop portion 31c is formed so that the band 6 can be passed therethrough and the band 6 can be fixed or the band 6 can be folded back. For example, the loop portion 31c is a member having a rectangular ring shape including an opening elongated in one direction through which the band 6 can be inserted. The loop portion 31c is integrally formed with an outer side surface of the outer case 31. One end of the band 6 is fixed to one loop portion 31c of the pair of loop portions 31c, and then the band 6 is folded back to the other loop portion 31c.

The windshield 32 is a glass plate having a shape similar to that of an outer peripheral edge of the outer case 31, and is a rectangular shape in the present embodiment. Note that the windshield 32 is not limited to a glass plate as long as the material has transparency or translucency.

The cuff cover 33 covers the bottom portion 31b of the outer case 31. In the cuff cover 33, an opening 33a is formed in which the projection portion 31b1 of the bottom portion 31b is disposed, and through which the projection portion 31b1 is exposed to the outside. For example, the cuff cover 33 is formed to a thickness and into a shape such that when the cuff cover 33 is fixed to the bottom portion 31b, the projection portion 31b1 projects from a main surface of the cuff cover 33 facing the wrist 300. Further, for example, the cuff cover 33 is formed with holes 33b in which screws are disposed at four corners, and is detachably fixed to the bottom portion 31b of the outer case 31 of the case 11 by screws or the like.

The cuff cover 33 covers end portions of the first cuff structure 4 and the second cuff structure 5 disposed on the bottom portion 31b, and thus the end portions of the first cuff structure 4 and the second cuff structure 5 are disposed in gaps formed between the cuff cover 33 and the bottom portion 31b, and the first cuff structure 4 and the second cuff structure 5 are fixed to the bottom portion 31b. As a specific example, the cuff cover 33 covers a part of the flow path body 72 and the connection portion 73 of the first pressing cuff 52 described later, a flow path body 82 and three connection portions 83 of a sensing cuff 54, and a flow path body 92 and two connection portions 93 of a second pressing cuff 62. Then, the cuff cover 33 fixes the first cuff structure 4 and the second cuff structure 5 by restricting movement of the covered portion of part of the first pressing cuff 52, the sensing cuff 54, and the second pressing cuff 62 in a direction away from the bottom portion 31b. Note that a configuration may be adopted in which the cuff cover 33 includes a buffer material between the first cuff structure 4 and the second cuff structure 5.

The display unit 12 is disposed directly below the windshield 32. The display unit 12 is electrically connected to the processor 23. The display unit 12 is, for example, a liquid crystal display or an organic electroluminescence display. As a specific example, the display unit 12 is an organic light-emitting diode (OLED). The display unit 12 displays various information including the date and time, measurement results of blood pressure values such as the systolic blood pressure and diastolic blood pressure, heart rate, and the like, and information such as the charge state and remaining charge of the battery 18. The display unit 12 is formed in, for example, the same shape as that of the windshield 32 or a shape slightly smaller than that of the windshield 32 in plan view.

The operation unit 13 is configured to enable input of a command from a user. The operation unit 13 includes, for example, a plurality of buttons 41 provided on the case 11, a sensor that detects operation of the buttons 41, and a touch panel 43 provided on the display unit 12 or the windshield 32. When operated by the user, the operation unit 13 converts a command into an electrical signal. The sensor and the touch panel 43 are electrically connected to the processor 23 and output electrical signals to the processor 23.

The pump 14 is, for example, a piezoelectric pump. The pump 14, for example, compresses air as a fluid and supplies the compressed air through the fluid circuit 24 to an air bag 71 of the first pressing cuff 52 described below and an air bag 81 of the sensing cuff 54 of the first cuff structure 4, and an air bag 91 of the second pressing cuff 62, to be described below, of the second cuff structure 5. The pump 14 is electrically connected to the processor 23.

The acceleration sensor 15 is, for example, a 3-axis acceleration sensor. The acceleration sensor 15 measures acceleration and outputs an analog signal, for example. The acceleration sensor 15 is connected to the processor 23 via, for example, an A/D conversion circuit.

The valve 16 is, for example, an open-close valve. The valve 16 opens and closes a fluid circuit connecting the pump 14 and the first cuff structure 4 and/or the second cuff structure 5, and/or a fluid circuit connecting the first cuff structure 4 and the outside (atmosphere) in the fluid circuit 24. The valve 16 is electrically connected to the processor 23. For example, the valve 16 is opened and closed by the control of the processor 23.

As a specific example, the valve 16 is a safety valve that releases the air supplied to the first pressing cuff 52, the second pressing cuff 62, and the sensing cuff 54, which will be described later, of the first cuff structure 4 to the atmosphere. For example, when air is supplied to the first pressing cuff 52 and the sensing cuff 54 at the time of blood pressure measurement, the valve 16 is switched to a closed state under the control of the processor 23. Further, when the air in the first pressing cuff 52 and the sensing cuff 54 is exhausted, the valve 16 is switched from the closed state to an open state under the control of the processor 23. Further, the valve 16 may be formed so that the opening degree is adjustable. The valve 16 may be provided on the fluid circuit 24, or may be integrally provided inside s housing of the pump 14.

The pressure sensor 17 is provided, for example, in the fluid circuit 24. The pressure sensor 17 detects the pressures of the first pressing cuff 52 and/or the sensing cuff 54. The pressure sensor 17 detects the pressure of the sensing cuff 54, for example.

The pressure sensor 17 is electrically connected to the processor 23 via an A/D conversion circuit, for example, converts the detected pressure into an electrical signal, and outputs the electrical signal to the processor 23.

The battery 18 is, for example, a secondary battery such as a rechargeable lithium ion battery. The battery 18 is electrically connected to the processor 23. The battery 18 supplies power to the processor 23. The battery 18 supplies power for driving to the respective configurations of the processor 23 and to the display unit 12, the operation unit 13, the pump 14, the acceleration sensor 15, the valve 16, the pressure sensor 17, the communication unit 19, and the biometric sensors 20 via the processor 23.

The communication unit 19 is configured to be capable of transmitting and receiving information to and from an external device wirelessly and/or by wire. The communication unit 19 is, for example, a wireless communication module conforming to a wireless communication standard. The communication unit 19, for example, transmits information, such as information controlled by the processor 23 and measured blood pressure values and pulse, to an external device, and receives a recording medium for software updates and the like from an external device and transmits the recording medium and the like to a control unit. In the present embodiment, the external device is, for example, an external terminal such as a smartphone, a tablet terminal, a personal computer, or a smart watch.

In the present embodiment, the communication unit 19 and the external terminal may be directly connected, or may be connected via a network. The communication unit 19 and the external terminal may be connected via a mobile communication network, such as 4G or 5G, and a wireless communication line, such as WiMAX or Wi-Fi (trade names). Further, the communication unit 19 and the external device may be connected by a wireless communication means, such as Bluetooth (trade name) Low Energy (BLE), near field communication (NFC), and infrared communication. Further, the communication unit 19 may include, for example, a general-purpose connector such as a micro-universal serial bus (USB) in addition to a wireless communication module, and a dedicated connector for the blood pressure measurement device 1, and may be connected to the external terminal directly or via a wired communication line such as a local area network (LAN) connection using various cables such as a USB cable. Thus, a configuration may be adopted in which the communication unit 19 includes a plurality of communication means, such as a wireless antenna and a micro-USB connector. Note that the connector for wired communication may be a dedicated connector for the blood pressure measurement device 1.

The biometric sensors 20 are sensors formed so as to be capable of detecting biological information by being in contact with or by facing the wrist 300. Each of the biometric sensors 20 converts the detected biological information into an electrical signal, and outputs the electrical signal to the processor 23. The biometric sensor 20 may be, for example, a sensor that measures a physical quantity such as a heart rate or a body temperature, or may be a sensor that measures a chemical value such as a blood sugar level or a blood oxygen concentration. In the present embodiment, the biometric sensors 20 include, for example, a PPG sensor 20a, an SpO2 sensor 20b, and an ECG sensor 20c.

For example, the PPG sensor 20a measures the heart rate by photoplethysmography. For example, the PPG sensor 20a includes a first LED 20d, a second LED 20e, and a first photodiode (PD) 20f.

For example, the SpO2 sensor 20b measures a saturation (transcutaneous arterial oxygen saturation). The SpO2 sensor 20b includes the second LED 20e and a second PD 20g. Here, for example, the PPG sensor 20a and the SpO2 sensor 20b share the second LED 20e.

For example, the ECG sensor 20c measures a flow of electricity in the heart to acquire an electrocardiogram waveform. For example, the ECG sensor 20c includes a pair of electrocardiographic electrodes 20c1.

The charging circuit unit 21 includes, for example, an antenna unit 211, a power-receiving unit 212, a charging unit 213, and the charging terminal 214. The charging circuit unit 21 charges the battery 18 by wire power transfer and/or wireless power transfer. For example, the charging circuit unit 21 receives transmission power transmitted by the antenna unit 211 from an antenna unit 103 of a power transmission device 100 externally provided, and charges the battery 18. Further, for example, the charging circuit unit 21 receives transmission power transmitted by the charging terminal 214 from a power transmission terminal 104 of the power transmission device 100 externally provided, and charges the battery 18. That is, the charging circuit unit 21 charges the battery 18 by selectively using the antenna unit 211 and the charging terminal 214 and receiving power from the power transmission device 100. Note that the charging circuit unit 21 may be formed so as to be capable of both wire power transfer and wireless power transfer or may be formed so as to be capable of only one of wire power transfer or wireless power transfer.

The antenna unit 211 receives transmission power from the antenna unit 103 of the power transmission device 100. The antenna unit 211 is, for example, a receiver coil as a power-receiving resonance circuit. The antenna unit 211 supplies the received power to the power-receiving unit 212. A power-receiving surface of the antenna unit 211 is formed in a planar shape. The antenna unit 211 is disposed, for example, in the case 11. As a specific example, the antenna unit 211 is provided adjacent to the display unit 12 on a side opposite to the windshield 32 of the display unit 12 in the case 11.

The antenna unit 211 includes, for example, a resonance capacitor and constitutes the power-receiving resonance circuit.

The power-receiving unit 212 rectifies power received by the antenna unit 211 or the charging terminal 214, and supplies the rectified power to the charging unit 213. As a specific example, the power-receiving unit 212 rectifies the received power supplied from the antenna unit 211 and converts the rectified power from alternating current (AC) to direct current (DC). For example, the power-receiving unit 212 includes a rectifying circuit and a control circuit, controls the operation of the rectifying circuit by the control circuit, and outputs rectified DC power to the charging unit 213.

The charging unit 213 supplies the electric power supplied from the power-receiving unit 212 to the battery 18 as electric power for charging. For example, the charging unit 213 converts the electric power supplied from the power-receiving unit 212 into a predetermined current value and a predetermined voltage value and supplies the converted electric power to the battery 18. Further, for example, the charging unit 213 may include a circuit that outputs the charge state of the battery 18 to the power-receiving unit 212 and/or the processor 23.

The charging terminal 214 includes, for example, a pair of terminals 214a, and receives transmission power from the power transmission terminal 104 of the power transmission device 100 via the pair of terminals 214a.

The memory 22 includes, for example, a random access memory (RAM) and a read only memory (ROM). The memory 22 stores various data. For example, the memory 22 pre-stores, in a changeable manner, for example, various recording medium data such as recording mediums and applications for controlling the blood pressure measurement device 1 in its entirety and the pump 14, settings data for setting various functions of the blood pressure measurement device 1, calculation data for calculating blood pressure values from the pressure measured by the pressure sensor 17, calculation data for calculating biological information such as a heart rate, a saturation, and an electrocardiogram waveform from the information measured by the biometric sensors 20.

The processor 23 controls the operation of the blood pressure measurement device 1 in its entirety, and the operations of the pump 14 and the valve 16 on the basis of the recording mediums stored in the memory 22, and executes a predetermined operation (function). Further, the processor 23 executes the predetermined calculation, analysis, processing, or the like according to the read recording medium. The processor 23 is an arithmetic device such as a CPU. The processor 23 may include, for example, a sub-CPU in addition to a main CPU. Further, the processor 23 displays a state or a result of the executed various operations, calculations, analyses, processing, and the like on the display unit 12 by a recording medium or an application.

The fluid circuit 24 fluidly connects at least two or more of the pump 14, the valve 16, the pressure sensor 17, the first cuff structure 4, and the second cuff structure provided in the case 11. The fluid circuit 24 may include, for example, a pipe and a flow path plate 24a forming a flow path of a fluid supplied from the pump 14 to the first cuff structure 4, components such as a single orifice 24b or a plurality of orifices 24b that are flow rate resistors for controlling a supply amount and a pressure of the fluid supplied to the first cuff structure 4 and/or the second cuff structure 5, and a check valve for controlling a flow direction of the fluid. The flow path plate 24a is formed by joining a plurality of plates made of a resin material or a metal material, and sheets or double-sided tapes disposed between the plates adjacent to each other and in which slits are formed to serve as a flow path. Note that the plurality of orifices 24b may be provided in the flow path plate 24a, or may be provided in a part of the first cuff structure 4 or a part of the second cuff structure 5.

The substrate 25 includes, for example, a control board 25a, a sensor main board 25b, and a sensor sub-board 25c.

On the control board 25a, for example, the acceleration sensor 15, the valve 16, the pressure sensor 17, the communication unit 19, the circuit configuration of the charging circuit unit 21, the memory 22, and the processor 23 are mounted.

On the sensor main board 25b, the first LED 20d, the second LED 20e, the first PD 20f, and the second PD 20g used for the PPG sensor 20a and the SpO2 sensor 20b are mounted.

On the sensor sub-board 25c, various circuits and electronic components for configuring the PPG sensor 20a, the SpO2 sensor 20b, and the ECG sensor 20c are mounted. For example, the control board 25a, the sensor main board 25b, and the sensor sub-board 25c are electrically connected to one another.

Hereinafter, a specific example of the first cuff structure 4 will be described with reference to FIGS. 1 to 3 and FIG. 5. As illustrated in FIGS. 1 to 3 and FIG. 5, the first cuff structure 4 includes a first curler 51, the first pressing cuff 52, a back plate 53, and the sensing cuff 54. The first cuff structure 4 includes, for example, the first curler 51, the first pressing cuff 52, the back plate 53, and the sensing cuff 54. The first cuff structure 4 is configured by sequentially layering the first curler 51, the first pressing cuff 52, the back plate 53, and the sensing cuff 54 toward the wrist 300. Note that a configuration may be adopted in which the first cuff structure 4 does not include the first curler 51 and/or the back plate 53.

The first curler 51 is, for example, fixed, at one end side, to the bottom portion 31b provided on the wrist 300 side of the case 11. The first curler 51 is formed in a band-like shape that curves in a shape following a circumferential direction of the wrist 300. The first curler 51 is constituted by a resin material. The first curler 51 is made of a material having a low hardness appropriate to provide flexibility and shape retainability. Here, “flexibility” refers to deformation of the shape of the first curler 51 in a radial direction at the time of application of an external force from the band 6 to the first curler 51. “Shape retainability” refers to the ability of the first curler 51 to maintain a pre-imparted shape when no external force is applied to the first curler 51. That is, the first curler 51 is made of a resin material having a hardness that does not cause compression deformation or that hardly causes compression deformation, but allows for elastic deformation such as bending deformation in which the shape, particularly, a curvature of a curved portion, is changed. Accordingly, the first curler 51 is formed elastically deformable so as to be bent and deformed by application of an external force and to increase or decrease the internal space in which the wrist 300 is disposed in accordance with the shape of the wrist on which the first curler 51 is worn. For example, the first curler 51 is made of a thermoplastic polyurethane resin (hereinafter referred to as TPU) or a polypropylene resin.

Further, the first curler 51 on one end side is fixed to the case 11. Further, the first curler 51 is formed to such a length so as to face at least one of two arteries 311 and 312 when the blood pressure measurement device 1 is worn on the wrist 300 having the longest circumference among the users who are expected to wear the device. Preferably, the first curler 51 is formed to such a length so as to face the two arteries 311 and 312 when the blood pressure measurement device 1 is worn on the wrist 300 having the longest circumference among the users who are expected to wear the device. Further, the first curler 51 is set to such a length that the other end of the first curler 51 does not come into contact with the device body 3 when the blood pressure measurement device 1 is worn on the wrist 300 having the shortest circumference among the users who are expected to wear the device. In the first curler 51, for example, a portion extending from the case 11 of the device body 3 is curved by a predetermined radius of curvature so as to conform to the shape of the side of one wrist 300 of the left and right wrists 300 and the shape of the palm side of the wrist 300.

The first pressing cuff 52 is fixed to an inner peripheral surface of the first curler 51 by double-sided tape, an adhesive, thermal welding, or the like. The first pressing cuff 52 is fluidly connected to the pump 14 through the fluid circuit 24. The first pressing cuff 52 on one main surface is fixed to an inner surface of the first curler 51. The first pressing cuff 52 is inflated, thereby pressing the back side of the wrist 300 and pressing the back plate 53 and the sensing cuff 54 toward the wrist 300.

The first pressing cuff 52 includes, for example, one or a plurality of air bags 71, the flow path body 72 provided at an end portion of the air bag 71, and the connection portion 73 such as a nipple provided in the flow path body 72 and connected to the fluid circuit 24. Here, the air bag 71 is a bag-like structure and, in the present embodiment, the blood pressure measurement device 1 is configured to use air with the pump 14, and thus the present embodiment will be described using the air bag. However, in a case in which a fluid other than air is used, the bag-like structure need only be any fluid bag that is inflated by the fluid. The air bag 71 is formed in a rectangular bag shape elongated in one direction.

The air bag 71 is made by forming a plurality of sheet members into a bag shape by thermal welding or the like. For example, when a configuration is adopted in which the first pressing cuff 52 includes a plurality of air bags 71, the plurality of air bags 71 are layered, integrally formed by welding or the like, and fluidly continuous. The sheet members forming the air bag 71 are made of a thermoplastic elastomer, for example. The thermoplastic elastomer is, for example, TPU.

The flow path body 72 is, for example, integrally provided at a part of one edge portion of the air bag 71 in a longitudinal direction. The flow path body 72 is formed by a portion of two sheet members of the plurality of sheet members forming the air bag 71. Further, the flow path body 72 is formed in a shape elongated in one direction with a width smaller than a width of the air bag 71 in a lateral direction. The flow path body 72 is integrally provided with the connection portion 73 at a tip end. The flow path body 72 is connected to the fluid circuit 24 via the connection portion 73 and constitutes a flow path between the fluid circuit 24 and the air bag 71. A thickness of the flow path body 72 at the time of inflation is smaller than a thickness of the air bag 71 at the time of inflation.

For example, one connection portion 73 is provided. The one connection portion 73 is connected to the fluid circuit 24 via one hole portion 31b3 formed in the bottom portion 31b.

Note that, for example, the flow path body 72 extends from one end side to the other end side of the bottom portion 31b in the opposing direction of the pair of loop portions 3 1c and adjacent to the projection portion 31b 1 in order to avoid the projection portion 31b1 formed in the bottom portion 31b, and the connection portion 73 is connected to one hole portion 31b3 of the three hole portions 31b3 formed at the end portion of the bottom portion 31b on the side where the second cuff structure 5 projects.

The back plate 53 is fixed to the surface of the first pressing cuff 52 on the wrist 300 side by double-sided tape, an adhesive, or the like. The back plate 53 is made of a resin material. The back plate 53 is formed in a rectangular plate shape elongated in one direction, for example. Note that the back plate 53 may, for example, be configured to be divided, that is, may be formed by aligning a plurality of small rectangular pieces in one direction. The back plate 53 has shape conformability.

This “shape conformability” refers to a function in which the back plate 53 can be deformed in such a manner as to conform to the shape of a contacted portion of the wrist 300 to be positioned. This contacted portion of the wrist 300 refers to a region of the wrist 300 that comes into contact with the back plate 53. This contact includes both direct contact with the back plate 53 and indirect contact with the back plate 53 with the sensing cuff 54 interposed therebetween.

The sensing cuff 54 is fixed to a main surface of the back plate 53 on the wrist side. The sensing cuff 54 comes into contact with a region of the wrist 300 where the arteries 311 and 312 are present, directly or indirectly with a cover or the like interposed therebetween. The sensing cuff 54 is formed into a rectangular shape elongated in one direction. Note that a configuration may be adopted in which the sensing cuff 54 comes into contact with a region of the wrist 300 where one of the arteries 311 and 312 is present. The sensing cuff 54 is the same size as the first pressing cuff 52 or smaller than the first pressing cuff 52 in the longitudinal direction. Further, the sensing cuff 54 is the same size as the first pressing cuff 52, smaller than the first pressing cuff 52, or larger than the back plate 53 in the lateral direction. The sensing cuff 54 is in the same shape as that of the back plate 53 or is smaller than the back plate 53, in the longitudinal direction and the width direction of the back plate 53. The sensing cuff 54 compresses the region of the wrist on the palm side where the artery is present by being inflated. The sensing cuff 54 is pressed by the inflated first pressing cuff 52 toward the living body with the back plate 53 interposed therebetween.

As a specific example, the sensing cuff 54 includes the air bag 81, the flow path body 82 fluidly connected to the air bag 81, and the connection portion 83 such as a nipple provided at the flow path body 82. The air bag 81 and the flow path body 82 are made by forming a plurality of sheet members into a bag shape by thermal welding or the like. The sheet members forming the air bag 81 and the flow path body 82 are made of, for example, a thermoplastic elastomer. The thermoplastic elastomer is, for example, TPU.

Here, the air bag 81 is a bag-like structure and, in the present embodiment, the blood pressure measurement device 1 is configured to use air with the pump 14, and thus the present embodiment will be described using the air bag. However, in a case in which a fluid other than air is used, the bag-like structure may be a fluid bag or the like that is inflated by the fluid. The air bag 81 is constituted in a rectangular shape elongated in one direction.

The flow path body 82 is, for example, integrally provided at a part of one edge portion of the air bag 81 in the longitudinal direction. The flow path body 82 is formed by a portion of two sheet members of the plurality of sheet members forming the air bag 81. Further, the flow path body 82 is formed in a shape elongated in one direction with a width smaller than a width of the air bag 81 in the lateral direction. The flow path body 82 is integrally provided with the connection portion 83 at a tip end. The flow path body 82 is connected to the fluid circuit 24 via the connection portion 83 and constitutes a flow path between the fluid circuit 24 and the air bag 81. A thickness of the flow path body 82 at the time of inflation is smaller than a thickness of the air bag 81 at the time of inflation.

For example, three connection portions 83 are provided. The three connection portions 83 are connected to the three hole portions 31b3 formed at the end portion of the bottom portion 31b on the side where the first cuff structure 4 projects. The three connection portions 83 are connected to the fluid circuit 24.

Hereinafter, a specific example of the second cuff structure 5 will be described with reference to FIGS. 1 to 3 and FIG. 5. As illustrated in FIGS. 1 to 3 and FIG. 5, the second cuff structure 5 includes a second curler 61 and the second pressing cuff 62. The second cuff structure 5 is configured by sequentially layering the second curler 61 and the second pressing cuff 62 toward the wrist 300.

The second curler 61 is, for example, fixed, at one end side, to the bottom portion 31b provided on the wrist 300 side of the case 11. The second curler 61 is formed in a band-like shape that curves in a shape following the circumferential direction of the wrist 300. The second curler 61 is constituted by a resin material. The second curler 61 is made of a material having a low hardness appropriate to provide flexibility and shape retainability. Here, “flexibility” refers to deformation of the shape of the second curler 61 in the radial direction at the time of application of an external force from the band 6 to the second curler 61. “Shape retainability” refers to the ability of the second curler 61 to maintain a pre-imparted shape when no external force is applied to the second curler 61. That is, the second curler 61 is made of a resin material having a hardness that does not cause compression deformation or that hardly causes compression deformation, but allows for elastic deformation such as bending deformation in which the shape, particularly the curvature of a curved portion, is changed. Accordingly, the second curler 61 is formed elastically deformable so as to be bent and deformed by application of an external force and to increase or decrease the internal space in which the wrist 300 is disposed in accordance with the shape of the wrist on which second curler 61 is worn. For example, the second curler 61 is made of a thermoplastic polyurethane resin (hereinafter referred to as TPU) or a polypropylene resin.

Further, the second curler 61 on one end side is fixed to the case 11. Further, the second curler 61 is formed to such a length so as to face at least one of the two arteries 311 and 312 and overlap the first cuff structure 4 at an end portion side when the blood pressure measurement device 1 is worn on the wrist 300 having the longest circumference among the users who are expected to wear the device. Here, one of the two arteries 311 and 312 is the radial artery 311 and the other is the ulnar artery 312. Preferably, the second curler 61 is formed to such a length so as to face the two arteries 311 and 312 when the blood pressure measurement device 1 is worn on the wrist 300 having the longest circumference among the users who are expected to wear the device. Further, the second curler 61 is set to such a length that the other end of the second curler 61 does not come into contact with the device body 3 when the blood pressure measurement device 1 is worn on the wrist 300 having the shortest circumference among the users who are expected to wear the device. In the second curler 61, for example, a portion extending from the case 11 of the device body 3 is curved with a predetermined radius of curvature so as to conform to the shape of the side of one wrist 300 of the left and right wrists 300 and the shape of the palm side of the wrist 300.

The second pressing cuff 62 is fixed to an inner peripheral surface of the second curler 61 by double-sided tape, an adhesive, thermal welding, or the like. The second pressing cuff 62 is fluidly connected to the pump 14 through the fluid circuit 24. The second pressing cuff 62 on one main surface is fixed to an inner surface of the second curler 61. The second pressing cuff 62 is inflated, thereby pressing the opposing wrist 300 and pressing the overlapped first cuff structure 4 toward the wrist 300. A thickness of the second pressing cuff 62 when inflated is the same as a thickness of the first pressing cuff 52 or greater than the thickness of the first pressing cuff 52 when inflated.

The second pressing cuff 62 includes, for example, one or a plurality of air bags 91, the flow path body 92 provided at an end portion of the air bag 91, and the connection portion 93 such as a nipple provided in the flow path body 92 and connected to the fluid circuit 24. Here, the air bag 91 is a bag-like structure, and in the present embodiment, the blood pressure measurement device 1 is configured to use air with the pump 14, and thus the present embodiment will be described using the air bag. However, in a case in which a fluid other than air is used, the bag-like structure need only be a fluid bag that is inflated by a fluid. The air bag 91 is formed in a rectangular bag shape elongated in one direction.

The air bag 91 is made by forming a plurality of sheet members into a bag shape by thermal welding or the like. For example, when a configuration is adopted in which the second pressing cuff 62 includes a plurality of air bags 91, the plurality of air bags 91 are layered, integrally formed by welding or the like, and fluidly continuous. The sheet members forming the air bag 91 are made of a thermoplastic elastomer, for example. The thermoplastic elastomer is, for example, TPU.

The flow path body 92 is, for example, integrally provided at a part of one edge portion of the air bag 91 in the longitudinal direction. The flow path body 92 is formed by a portion of two sheet members of the plurality of sheet members forming the air bag 91. Further, the flow path body 92 is formed into a shape elongated in one direction with a width smaller than a width of the air bag 91 in the lateral direction. The flow path body 92 is integrally provided with the connection portion 93 at a tip end. The flow path body 92 is connected to the fluid circuit 24 via the connection portion 93 and constitutes a flow path between the fluid circuit 24 and the air bag 91. A thickness of the flow path body 92 at the time of inflation is smaller than a thickness of the air bag 91 at the time of inflation.

For example, two connection portions 93 are provided. The two connection portions 93 are connected to the fluid circuit 24 via two hole portions 31b3 formed in the bottom portion 31b.

Note that for example, the connection portion 93 is connected to two hole portions 31b3 of the three hole portions 31b3 formed at the end portion of the bottom portion 31b on the side where the second cuff structure 5 projects.

The first pressing cuff 52 and the second pressing cuff 62 of the first cuff structure 4 and the second cuff structure 5 configured in this manner are formed to have such a length that when the blood pressure measurement device 1 is worn on the wrist 300 having the maximum circumference, an inflated portion of at least one of the air bag 71 of the first pressing cuff 52 and the air bag 91 of the second pressing cuff 62 covers at least one of the two arteries 311 and 312, and at least a part of the air bag 91 can overlap at least a part of the air bag 71. Further, the first cuff structure 4 and the second cuff structure 5 are formed to have such a length that the end portion does not come into contact with the device body 3 when the blood pressure measurement device 1 is worn on the wrist 300 having the minimum circumference.

The band 6 is formed in a belt-like shape on an outer peripheral side of the first cuff structure 4 and the second cuff structure 5 on the side opposite to the wrist 300. The band 6 is not fixed to the first cuff structure 4 or the second cuff structure 5. The band 6 is fixed at one end portion to one loop portion 31c. Further, the band 6 includes a pair of hook-and-loop fasteners 6a on one of which hooks are formed, and on the other of which loops are formed. The band 6 with an end portion side inserted into the other loop portion 31c is fixed by engaging the hooks and the loops with each other. Further, the band 6 includes, at the other end portion, a grip 6b that facilitates gripping by the user.

For example, the band 6 is fixed to the loop portion 31c formed on the side from which the second cuff structure 5 extends, is inserted into the loop portion 31c formed on the side from which the first cuff structure 4 extends, and is then folded back. The band 6 has such a length that, when the blood pressure measurement device 1 is worn on the wrist 300 having an expected maximum circumferential length, the band 6 can be inserted into the loop portion 31c provided at the outer case 31. Further, the hook-and-loop fastener 6a is provided on the band 6 with such a length and in such an arrangement that the band 6 can be folded back and fastened on the wrist 300 having an expected maximum circumferential length or an expected minimum circumferential length. The end portion of the band 6 folded back at the loop portion 31c is pulled in a direction away from the loop portion 31c, so that the blood pressure measurement device 1 is worn on the wrist 300 in a state in which the first cuff structure 4 and the second cuff structure 5 are wound around the wrist 300 as illustrated in FIG. 3. The band 6 is fixed by the hook-and-loop fastener 6a, restricting the first pressing cuff 52 and the second pressing cuff 62, when inflated, from expanding outwardly away toward a side opposite to the wrist, and thus making it possible for the first pressing cuff 52 and the second pressing cuff 62 to compress the wrist. This enables blood pressure measurement by a known oscillometric method.

Next, an example of a fluid configuration of the device body 3, the first cuff structure 4, the second cuff structure 5, and the fluid circuit 24 of the blood pressure measurement device 1 configured as described above will be described with reference to FIG. 10.

The first pressing cuff 52 is connected to the pump 14 via the flow path plate 24a, for example. The first pressing cuff 52 and the pump 14 are connected to the valve 16 via the flow path plate 24a, for example. The first pressing cuff 52 is connected to the second pressing cuff 62 via the flow path plate 24a, for example. The first pressing cuff 52 is connected to the sensing cuff 54 via, for example, the flow path plate 24a and the orifice 24b provided on the flow path plate 24a. The sensing cuff 54 is connected to the pressure sensor 17 via the flow path plate 24a, for example. The sensing cuff 54 is connected to the atmosphere via, for example, the flow path plate 24a and the orifice 24b provided on the flow path plate 24a. Here, the orifice 24b between the first pressing cuff 52 and the sensing cuff 54, and the orifice 24b between the sensing cuff 54 and the atmosphere are set to flow rate resistors at which the first pressing cuff 52 and the sensing cuff 54 have a desired pressure difference.

Next, an example of the power transmission device 100 that transmits power to the charging circuit unit 21 of the device body 3 will be described. As illustrated in FIG. 4, the power transmission device 100 includes a power source 101, a power transmission unit 102, the antenna unit 103, and the power transmission terminal 104. Note that a configuration may be adopted in which the power transmission device 100 includes one of the antenna unit 103 or the power transmission terminal 104. The power source 101 is, for example, an AC adapter connected to a commercial power source. The power source 101 converts AC power input from the commercial power source into DC power and supplies the DC power to the power transmission unit 102.

The power transmission unit 102 generates AC power as transmission power from the DC power supplied from the power source 101, and supplies the AC power to the antenna unit 103. For example, the power transmission unit 102 generates AC power having a frequency that is the same or substantially the same as a resonance frequency of the power transmission resonance circuit of the antenna unit 103.

The antenna unit 103 is, for example, a transmitter coil of a power transmission resonance circuit. A power transmission surface of the antenna unit 103 is formed into a planar shape. The antenna unit 103 transmits power to the antenna unit 211 of the device body 3. The antenna unit 103 includes, for example, a resonance capacitor and constitutes the power transmission resonance circuit.

The power transmission terminal 104 comes into contact with the charging terminal 214 provided in the device body 3 and is formed so as to be fixable to the charging terminal 214.

In the blood pressure measurement device 1 configured in this manner, the first cuff structure 4 and the second cuff structure 5, which extend from the device body 3 and are fluidly connected to each other, are arranged on both sides of the device body 3 in one direction. Further, the device body 3 includes, at the bottom portion 31b of the case 11 that is the rear surface side, the projection portion (sensor unit) 31b1 as a space in which at least one of the biometric sensors 20 or the charging terminal 214 is disposed at a position away from the first cuff structure 4 and the second cuff structure 5, for example, on a center side of the bottom portion 31b. Accordingly, the first cuff structure 4, the second cuff structure 5, and at least one of the biometric sensors 20 or the charging terminal 214 can each be disposed on the rear surface side of the device body 3.

Further, the first cuff structure 4 and the second cuff structure 5 are fluidly connected to each other through the fluid circuit 24 including the flow path plate 24a. Note that the first cuff structure 4 and the second cuff structure 5 may directly fluidly connect the first pressing cuff 52 and the second pressing cuff 62. With these configurations, even when the first cuff structure 4 and the second cuff structure 5 are provided and fixed to the case 11 away from each other in one direction, it is still possible for the first cuff structure 4 and the second cuff structure 5 to be fluidly continuous.

Further, when the blood pressure measurement device 1 is worn on the wrist 300 having an expected maximum circumferential length, the air bag 71 of the first pressing cuff 52 covers the region where at least one artery of the two arteries 311 and 312 is present, and the air bag 91 of the second pressing cuff 62 can overlap at least a part of the air bag 71 of the first pressing cuff 52.

Thus, the wrist 300 can be pressed by the first pressing cuff 52 and the second pressing cuff 62 and, even if the air bag 71 of the first pressing cuff 52 and the air bag 91 of the second pressing cuff 62 are not disposed at the rear surface of the device body 3, a sufficient pressing force for compressing the artery can be secured.

Thus, when the blood pressure measurement device 1 is worn on the wrist 300 having an expected maximum circumferential length, the air bag 71 of the first pressing cuff 52 and the air bag 91 of the second pressing cuff 62 can overlap by a predetermined length. Further, when the blood pressure measurement device 1 is worn on the wrist 300 having the minimum circumferential length, the first cuff structure 4 and the second cuff structure 5 each have such a length as not to come into contact with the device body 3. Further, the first cuff structure 4 and the second cuff structure 5 are not directly joined to the band 6.

With these configurations, the blood pressure measurement device 1, with the air bags 71 and 91 of the first pressing cuff 52 and the second pressing cuff 62 being inflated in an overlapping manner, makes it possible to suitably press the artery by the first pressing cuff 52 and the second pressing cuff 62. Thus, the blood pressure measurement device 1 makes it possible to dispose the biometric sensors 20 on the rear surface side of the device body 3 and, even if the air bags 71 and 91 of the first pressing cuff 52 and the second pressing cuff 62 are not disposed on the rear surface of the device body 3, measure blood pressure with high accuracy.

Further, the case 11 includes the vent 31b6 covered with the waterproof moisture-permeable sheet 31b7 at the bottom portion 31b, facilitating ventilation in the case 11. Further, the vent 31b6 and the first cuff structure 4 as well as the second cuff structure 5 are disposed at positions away from each other in a portion of the bottom portion 31b other than the projection portion 31b1, making it possible to prevent the vent 31b6 from being closed by the first cuff structure 4 and the second cuff structure 5. Further, the vent 31b6 is provided at a position covered with the cuff cover 33 spaced apart from the bottom portion 31b, thereby protecting the vent 31b6 with the cuff cover 33. Thus, the blood pressure measurement device 1 can prevent dirt from adhering to the vent 31b6 and the waterproof moisture-permeable sheet 31b7, making it possible to prevent impairment of the ventilation function.

Further, the first cuff structure 4 and the second cuff structure 5 are covered with the cuff cover 33 at their end portions disposed at the bottom portion 31b of the case 11, and are thus restricted in movement in a direction away from the bottom portion 31b by the cuff cover 33. This makes it possible to prevent the first cuff structure 4 and the second cuff structure 5 from being readily detached from the device body 3.

Further, a configuration is adopted in which the blood pressure measurement device 1 is provided with various sensors 20a to 20c as the biometric sensors 20 and the charging terminal 214 at the bottom portion 31b of the device body 3. Thus, the device body 3 enables simplification of wiring and the like, increasing the degree of freedom in arrangement, by not requiring the biometric sensors 20 and the charging terminal 214 to be provided at a position far from the device body from the substrate 25 provided inside the case 11. Further, the biometric sensors 20 are provided in the case 11 having high rigidity, and can stably come into contact with the wrist 300 when the blood pressure measurement device 1 is worn on the wrist 300, making it possible to suitably acquire biological information by the biometric sensors 20. Further, the charging terminal 214 is provided at the bottom portion 31b and thus, when the blood pressure measurement device 1 is removed from the wrist 300, the charging terminal 214 is exposed to the outside, facilitating charging using the charging terminal 214.

Further, in the blood pressure measurement device 1, the first pressing cuff 52 and the second pressing cuff 62 are fluidly connected through the fluid circuit 24 provided inside the device body 3, making it possible to simplify the arrangement of the first pressing cuff 52 and the second pressing cuff 62 at the bottom portion 31b.

In the blood pressure measurement device 1 configured in this manner, the measurement accuracy is improved even if the biometric sensors 20 are disposed on the rear surface of the device body 3.

Note that the present invention is not limited to the embodiment described above. For example, in the example described above, the configuration has been described in which the first pressing cuff 52 of the first cuff structure 4 and the second pressing cuff 62 of the second cuff structure 5 are connected to, for example, the flow path plate 24a of the fluid circuit 24 via the flow path bodies 72 and 92 and the connection portions 73 and 93 formed respectively, and are fluidly connected in the fluid circuit 24.

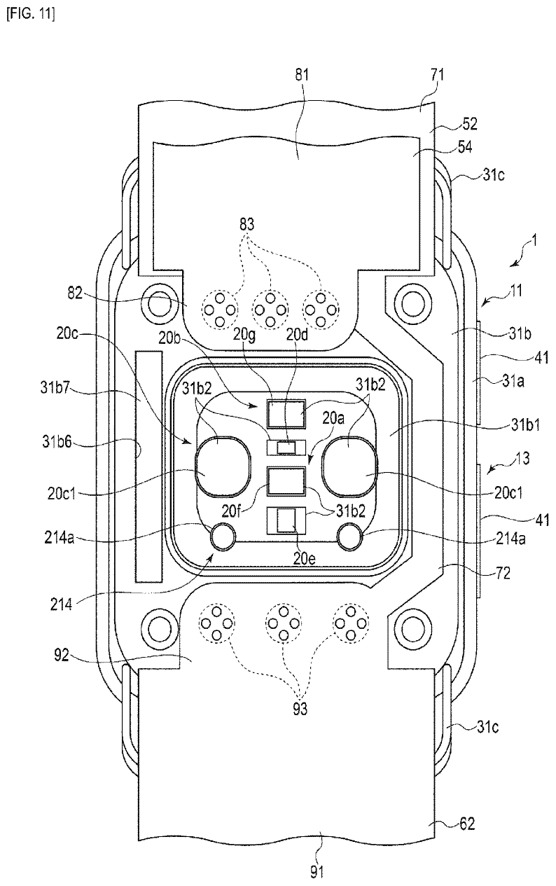

However, the first pressing cuff 52 and the second pressing cuff 62 may be configured to be fluidly and directly connected. For example, as illustrated in FIG. 11, the first cuff structure 4 and the second cuff structure 5 may have a configuration in which the flow path body 72 of the first pressing cuff 52 and the flow path body 92 of the second pressing cuff 62 are fluidly and integrally continuous and are connected to the fluid circuit 24 by the connection portion 93 formed in the flow path body 72 or the flow path body 92. With such a configuration, the first pressing cuff 52 and the second pressing cuff 62 are connected to each other, while avoiding the projection portion 31b1 which is a sensor unit. Thus, it is possible to prevent the first pressing cuff 52 and the second pressing cuff 62 from interfering with the projection portion 31b1 and hindering the functions of the biometric sensors 20 and the charging terminal 214 provided in the projection portion 31b1.

In the example described above, the configuration has been described in which the first cuff structure 4 includes the first curler 51, the first pressing cuff 52, the back plate 53, and the sensing cuff 54, and the second cuff structure 5 includes the second curler 61 and the second pressing cuff 62.

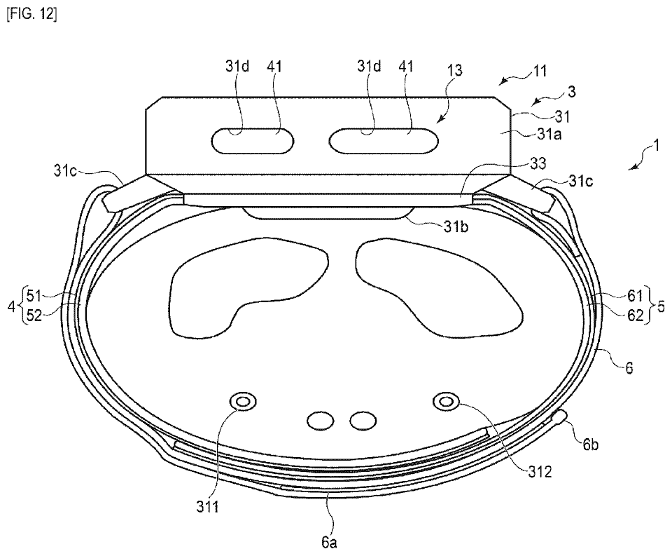

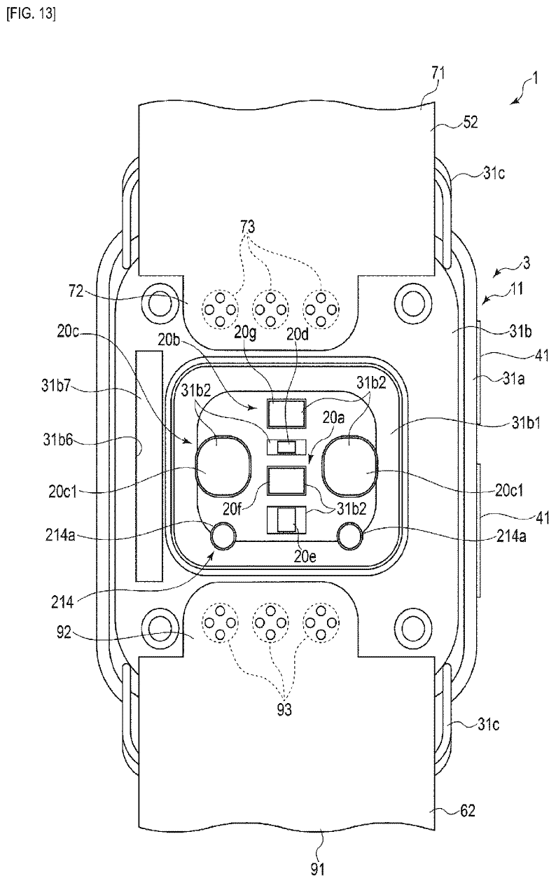

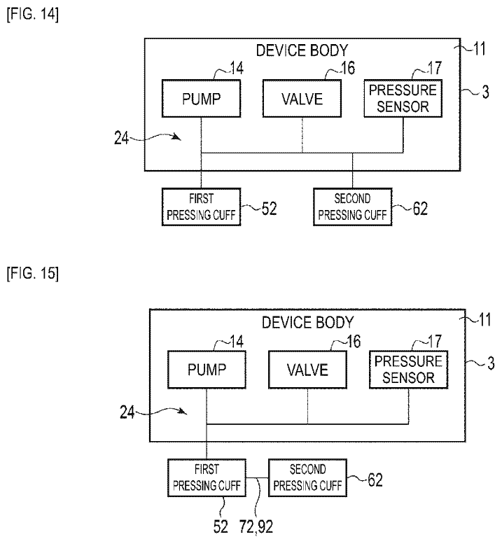

However, the first cuff structure 4 may not include at least one of the first curler 51 and the back plate 53, and the second cuff structure 5 may not include the second curler 61. Further, as illustrated in FIGS. 12 and 13, the first cuff structure 4 may not include the sensing cuff 54. For example, in a case in which the first cuff structure 4 does not include the sensing cuff 54, as illustrated in FIGS. 14 and 15, a configuration may be adopted in which the pressure sensor 17 is fluidly connected to at least the first pressing cuff 52 to obtain the blood pressure from the pressure of the first pressing cuff 52. Further, also in the case in which the first cuff structure 4 does not include the sensing cuff 54, the first pressing cuff 52 and the second pressing cuff 62 may be fluidly connected to each other through the fluid circuit 24 in the device body 3 as illustrated in FIG. 14, or the flow path body 72 and the flow path body 92, for example, may be fluidly connected as illustrated in FIG. 15.

Further, in the example described above, the band 6 is not fixed to the first cuff structure 4 or the second cuff structure 5. However, a configuration may be adopted in which the band 6 is fixed to the second cuff structure 5 which is on the outer side when the first cuff structure 4 and the second cuff structure 5 overlap. That is, even though the band 6 is fixed to the second cuff structure 5, when the first cuff structure 4 closer to the wrist 300 than the second cuff structure 5 is not fixed to the band 6, and when the band 6 is tightened, then the first cuff structure 4 and the second cuff structure 5 can be tightened and brought into close contact with the wrist 300.

Further, in the example described above, the configuration is adopted in which the device body 3 is provided with the PPG sensor 20a, the SpO2 sensor 20b, and the ECG sensor 20c as the biometric sensors 20 at the bottom portion 31b of the outer case 31 forming the rear surface side of the case 11. However, a configuration may be adopted in which the biometric sensor 20 is any one of the PPG sensor 20a, the SpO2 sensor 20b, and the ECG sensor 20c, or includes a sensor that acquires other biological information in addition to these sensors 20a to 20c or in place of these sensors 20a to 20c.

That is, the present invention is not limited to the above-described embodiments, and various modifications can be made in an implementation stage without departing from the gist thereof. Furthermore, each of the embodiments may be carried out as appropriate in combination to the extent possible, and combined effects can be obtained in such a case. Furthermore, the inventions at various stages are included in the above-described embodiments, and the various inventions can be extracted in accordance with appropriate combinations of the plurality of disclosed constituent elements. Note that the present invention is not limited to the above-described embodiments, and various modifications can be made in an implementation stage without departing from the gist. Further, embodiments may be combined as appropriate and carried out, and combined effects can be obtained in such a case. Further, the various inventions are included in the embodiment, and the various inventions may be extracted in accordance with combinations selected from the plurality of disclosed constituent elements. For example, in a case in which the problem can be solved and the effects can be obtained even when some constituent elements are removed from the entire constituent elements described in the embodiment, the configuration obtained by removing these constituent elements may be extracted as an invention.

### REFERENCE NUMERALS LIST

1 Blood pressure measurement device
3 Device body
4 First cuff structure
5 Second cuff structure
6 Band
6a Hook-and-loop fastener
6b Grip
11 Case
12 Display unit
13 Operation unit
14 Pump
15 Acceleration sensor
16 Valve
17 Pressure sensor
18 Battery
19 Communication unit
20 Biometric sensor
20a PPG sensor
20b SpO2 sensor
20c ECG sensor
20c1 Electrocardiographic electrode
20d First LED
20e Second LED
20f First PD
20g Second PD
21 Charging circuit unit
22 Memory
23 Processor
24 Fluid circuit
24a Flow path plate
24b Orifice
25 Substrate
25a Control board
25b Sensor main board
25c Sensor sub-board
31 Outer case
31a Peripheral wall portion
31b Bottom portion (rear surface)
31b1 Projection portion (sensor unit)
31b1 Sensor unit
31b2 Window portion
31b3 Hole portion
31b4 Opening
31b5 Cover
31b6 Vent
31b7 Waterproof moisture-permeable sheet
31c Loop portion
31d Opening
32 Windshield
33 Cuff cover
33a Opening
33b Hole
41 Button
43 Touch panel
51 First curler
52 First pressing cuff
53 Back plate
54 Sensing cuff
61 Second curler
62 Second pressing cuff
71 Air bag
72 Flow path body
73 Connection portion
81 Air bag
82 Flow path body
83 Connection portion
91 Air bag
92 Flow path body
93 Connection portion
100 Power transmission device
101 Power source
102 Power transmission unit
103 Antenna unit
104 Power transmission terminal
211 Antenna unit
212 Power-receiving unit
213 Charging unit
214 Charging terminal
214a Terminal
300 Wrist
311 Radial artery
312 Ulnar artery

What is claimed is:

1. A blood pressure measurement device to be worn on a wrist by a band, the device comprising:
a device body including a case, a pump provided in the case, a pressure sensor, a fluid circuit to be connected to the pump and the pressure sensor, and a sensor unit provided on a rear surface of the case facing toward the wrist;
a first pressing cuff connected to the fluid circuit and fixed to one end of the rear surface of the case in one direction away from the sensor unit;
a second pressing cuff connected to the fluid circuit, fixed to the other end of the rear surface of the case in the one direction away from the sensor unit, and fluidly connected to the first pressing cuff, and
a sensing cuff disposed on a side of one of the first pressing cuff and the second pressing cuff, the side facing toward the wrist, the sensing cuff being connected to the fluid circuit, wherein:
the first pressing cuff and the second pressing cuff are wound around the wrist by the band,
the first pressing cuff and the second pressing cuff are fluidly connected to each other through the fluid circuit provided in the device body,
when the blood pressure measurement device is worn on a wrist having an expected maximum circumference, at least one of the first pressing cuff and the second pressing cuff faces at least one of a radial artery and an ulnar artery passing through the wrist, and the first pressing cuff and the second pressing cuff overlap, and
the first pressing cuff and the second pressing cuff are connected to the fluid circuit at an end portion of the rear surface of the case on one side in the one direction, and the sensing cuff is connected to the fluid circuit at an end portion of the rear surface of the case on the other side in the one direction.

2. The blood pressure measurement device according to claim 1, wherein the sensor unit includes at least one of a biometric sensor and a charging terminal.

3. The blood pressure measurement device according to claim 2, wherein the first pressing cuff and the second pressing cuff are fluidly connected to each other on the rear surface, while avoiding the sensor unit.

4. The blood pressure measurement device according to claim 1, comprising a cuff cover fixed to the rear surface, having an opening formed for disposing the sensor unit, and covering a part of the first pressing cuff and a part of the second pressing cuff.

5. The blood pressure measurement device according to claim 4, wherein the device body includes a vent formed at a position on the rear surface away from the sensor unit.

6. The blood pressure measurement device according to claim 1, wherein the sensor unit includes at least one of a PPG sensor, an SpO2 sensor, and an electrocardiographic electrode.
